(19) United States
(12) Patent Application Publication
(10) Pub. No.: US 2020/0261717 A1
(43) Pub. Date: Aug. 20, 2020

(54) Method of Enhancing the Alternative Cellular Energy Pathway in Humans and Animals Using Wearable Items That Contain KELEA Activated Water

(71) Applicant: William John Martin, South Pasadena, CA (US)

(72) Inventor: William John Martin, South Pasadena, CA (US)

(21) Appl. No.: 16/278,712

(22) Filed: Feb. 19, 2019

(51) Int. Cl. A61N 1/10; G01N 13/02; G01N 5/02

(52) U.S. Cl. CPC A61N 1/10; G01N 5/02; G01N 13/02

(57) ABSTRACT

Placing a mini hot water bottle or other suitable container of an energized form of water referred to as KELEA activated water can be used to support the alternative cellular energy (ACE) pathway in a human or animal subject. KELEA is an abbreviation of kinetic energy limiting electrostatic attraction. The ACE pathway is a form of cellular energy different from that provided by the calories in food or, in the case of plants and certain bacteria, by photosynthesis. The long-term wearing of a flexible container of KELEA activated water, also referred to as waterceutical, provides many advantages over the transient uses of waterceuticals as drinking water, injection, spray or inhalant. Adding to the reliance and utility of the wearable waterceuticals is a method based on KELEA mediated weight gain of paper and the reversal of the weight gain by electromagnetic radiation. A practical wearable waterceutical using any of several suggested waterceutical products is described.

### RELATED PUBLICATION BY THE APPLICANT

1. Martin, W. J. (2003) Stealth Virus Culture Pigments, A Potential Source of Cellular Energy. Experimental Molecular Pathology, 74, 210-223.
2. Martin, W. J. (2005) Alternative Cellular Energy Pigments from Bacteria of Stealth Virus Infected Individuals. Experimental Molecular Pathology, 78, 217-217.
3. Martin W J (2014). Stealth Adapted Viruses; Alternative Cellular Energy (ACE) & KELEA Activated Water. Author House, IN. pp 321. ISBN 978-1-4969-0496-6.
4. Martin, W. J. (2014) KELEA Activated Water—Enhancing the Alternative Cellular Energy (ACE) Pathway. In Stealth Adapted Viruses; Alternative Cellular Energy (ACE) & KELEA Activated Water. Author House, Bloomington, Ind., USA, pp. 115-144.
5. Martin, W. J. (2014) KELEA Activated Water Leading to Improved Quantity & Quality of Agricultural Crops. Advances in Plants & Agriculture Research, 2, 00033.
6. Martin, W. J. (2015) Therapeutic Potential of KELEA Activated Water. International J of Complementary & Alternative Medicine, 1(1), 00001.
7. Martin, W. J. (2015) Alternative Cellular Energy Pathway Therapy Using KELEA Activated Water. International J Complementary & Alternative Medicine, 2(2), 00051.
8. Martin, W. J. (2015) KELEA, A Natural Energy That Seemingly Reduces Intermolecular Hydrogen Bonding in Water and Other liquids. Open Journal of Biophysics, 5, 69-79.
9. Martin, W. J. and Laurent, D. (2015) Homeopathy as A Misnomer for Activation of the Alternative Cellular Energy Pathway, Evidence for the Therapeutic Benefits of Enercel in a Diverse Range of Clinical Illnesses. International J Complementary & Alternative Medicine, 2(1), 00045
10. Martin, W. J. (2015) Interacting Light Paths Attract KELEA (Kinetic Energy Limiting Electrostatic Attraction) and Can Lead to the Activation of Water. Open Journal of Biophysics, 5, 115-121.
11. Martin, W. J. (2015) Interactive Electric Fields Can Attract KELEA (Kinetic Energy Limiting Electrostatic Attraction) and Can Lead to the Activation of Water. International J Complementary & Alternative Medicine, 1(6), 00034.
12. Martin, W. J. (2016) KELEA (Kinetic Energy Limiting Electrostatic Attraction) May Add to the Measured Weight of an Object. J Modern Physics, 7(6), 461-472.
13. Martin, W. J. (2016) KELEA (Kinetic Energy Limiting Electrostatic Attraction) Offers an Alternative Explanation to Existing Concepts Regarding Wave-Particle Duality, Cold Fusion and Superconductivity. J Modern Physics, 7(15), 1995-2007
14. Martin, W. J. (2016) Preparing and Using KELEA Activated Water to Enhance the Alternative Cellular Energy (ACE) Pathway in the Therapy of Multiple Illnesses. International J Complementary & Alternative Medicine, 3(1), 00059.
15. Martin, W. J. (2015) KELEA Activation of Water and Other Fluids for Health, Agriculture and Industry. Journal of Water Resource and Protection, 7, 1331-1344.
16. Martin, W. J. (2016) KELEA, Cosmic Rays, Cloud Formation and Electromagnetic Radiation, Electropollution as a Possible Explanation for Climate Change. Atmospheric and Climate Sciences, 6(2), 174-179.
17. Martin, W. J. (2016) Cancer as an Insufficiency of Cellular Energy (ICE), Therapeutic Approaches Based on Enhancing the Alternative Cellular Energy (ACE) Pathway. International J Complementary & Alternative Medicine, 3(3), 00074.
18. Martin, W. J. (2016) Insufficiency of Cellular Energy (ICE) in Neurons, From Electrical Hyperactivity to Quiescence. International Journal Complementary Alternative Medicine, 4, 00118.
19. Martin, W. J. (2016) Insufficiency of Cellular Energy (ICE) May Precede Neurodegeneration in Alzheimer's Disease and Be Treatable via the Alternative Cellular Energy (ACE) Pathway. Advances in Alzheimer's Disease, 6(1), 1-12.
20. Martin, W. J. (2016) Insufficiency of Cellular Energy (ICE), The Basis for Many Illnesses Potentially Correctable Using KELEA Activated Water. International J Complementary & Alternative Medicine, 4(1), 00106.
21. Martin, W. J. (2017) Cancer Is Treatable via the Alternative Cellular Energy (ACE) Pathway, Journal of Cancer Therapy, 8, 1279-1290
22. Martin, W. J. (2017) Using KELEA (Kinetic Energy Limiting Electrostatic Attraction) to Improve the Efficiency of Fuel Combustion. Open Journal Air Pollution, 6(3), 103-116.
23. Martin, W. J. (2017) Is KELEA (Kinetic Energy Limiting Electrostatic Attraction) a Source of Chemical Energy? MOJ Biorg Org Chem, 1(2), 54-58.
24. Martin, W. J. (2017) Insufficiency of Cellular Energy (ICE) from the Alternative Cellular Energy (ACE) Pathway Limiting the Specialized Functions of Neuronal Cells. International J Complementary & Alternative Medicine, 4(2), 00112.
25. Martin, W. J. (2017) Hyper-Excitability Followed by Functional Quiescence in Neuronal Cells Caused by Insufficient Cellular Energy (ICE), Treatable by Enhancing the Alternative Cellular Energy (ACE) Pathway. World Journal of Neuroscience, 7(3), 257-266.
26. Martin, W. J. (2017) Tissue Regeneration without Scarring Achieved by Enhancing the Alternative Cellular Energy (ACE) Pathway. Journal of Cosmetics, Dermatological Sciences and Applications, 7(1), 82-98.
27. Martin W J (2018) Is the placebo effect mediated by the Alternative Cellular Energy (ACE) pathway? International J Complementary & Alternative Medicine, 11(4): 231-233.
28. Martin W J (2018) KELEA Activated Water as an Alternative to Stem Cell Injections in Regenerative Medicine. International J Complementary & Alternative Medicine, 11(5), 251-258.
29. Martin W J (2019) Electromagnetic Radiation Causes Weight Loss and Weight Destabilization of Objects with Presumed Elevated Levels of KELEA (Kinetic Energy Limiting Electrostatic Attraction), Relevance to Human Health and to Global Warming. Modern J Physics (submitted)

### RELATED PATENT APPLICATIONS BY THE APPLICANT

ACE-pigments and Humic acid as energy sources. Application Ser. No. 10/192,936 Method of assessing and of activating the alternative cellular energy (ACE) pathway in the therapy of diseases. William John Martin Submitted Jan. 16, 2008. Application Ser. No. 12/009,195

Enerceutical mediated activation of the alternative cellular energy (ACE) pathway in the therapy of diseases. Application Ser. No. 12/151,779

Regenerative wound healing using copper-silver citrate composition. Application Ser. No. 12/288,749

Enerceutical activation of the alternative cellular energy (ACE) pathway in therapy of diseases. Application Ser. No. 12/069,597

Method of using the body's alternative cellular energy pigments (ACE-pigments) in the therapy of diseases Application Ser. No. 12/378,934

Urine as a source of alternative cellular energy pigments (ACE-pigments) in the assessment and therapy of diseases. Application Ser. No. 12/381,003

Activation of the alternative cellular energy (ACE) pathway in the therapy of diseases. Application Ser. No. 12/802,605

Methods for the detection of alternative cellular energy (ACE) pigments and for monitoring of the ACE pathway in the diagnosis and therapy of diseases. Application Ser. No. 12/802,763

Diagnostic value of systemic ACE pathway activation in the detection by fluorescence of localized pathological lesions. Application Ser. No. 12/804,619

Enerceutical mediated activation of the alternative cellular energy (ACE) pathway in the therapy of diseases. Application Ser. No. 12/151,779

Energy Charged Liquids to Enhance Enerceutical Activation of the Alternative Cellular Energy (ACE) Pathway in the Therapy of Diseases. Application Ser. No. 12/972,344

Energy Charged Alcoholic Beverages for Enhancing the Alternative Cellular Energy Pathway in the Prevention and Therapy of Diseases. Application Ser. No. 12/984,582

Energy Charged Liquids to Enhance Enerceutical Activation of the Alternative Cellular Energy (ACE) Pathway in the Therapy of Diseases Application Ser. No. 12/972,344

Methods for Detecting and Monitoring the Activity of Energized Water and Other Liquids Useful for Enhancing the Alternative Cellular Energy Pathway in the Prevention and Therapy of Diseases. Application Ser. No. 13/016,948

Methods for Increasing the Kinetic Activity of Alcohol, Water and Other Liquids, so as to Render the Liquids More Useful in Enhancing the Alternative Cellular Energy Pathway in the Prevention and Therapy of Diseases. Application Ser. No. 13/029,116

Methods for Increasing the Kinetic Activity of Alcohol, Water and Other Liquids, so as to Render the Liquids More Capable of Enhancing the Alternative Cellular Energy Pathway in the Prevention and Therapy of Diseases Application Ser. No. 13/040,262

Methods for Increasing the Kinetic Activity of Water and Other Liquids, so as to Render the Liquids More Useful in Enhancing the Alternative Cellular Energy Pathway and in Various Other Agricultural and Industrial Applications. Application Ser. No. 13/166,800

Use of Plants Extracts to Activate Water, Alcohol and Other Liquids. Submitted Oct. 27, 2011. Application Ser. No. 13/272,215.

Methods of Transferring Energies to Water, Alcohols and Minerals. Submitted Nov. 25, 2011. application Ser. No. 13/304,558.

Use of Certain Foods and Dietary Supplements as Water and Beverage Activating Enerceuticals. Application Ser. No. 14/507,822

Heat as a Method to Enhance the Fluid Activating Ability of Humic Acids, Zeolites and related Enerceuticals. Application Ser. No. 14/294,076

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not Applicable

### FIELD OF THE INVENTION

The invention is within the field of energy-based medicine. The research of the Applicant goes beyond the normal understanding and practice of energy medicine by clearly defining an alternative cellular energy (ACE) pathway that is driven by a novel form of energy, which has been defined by the Applicant as KELEA (kinetic energy limiting electrostatic attraction). These topics are embodied in the above cited publications and multiple submitted patent applications, which are incorporated herein by reference. The Applicant has argued that the natural environmental level of KELEA has been reduced below optimal levels because of increasing manmade electromagnetic radiation. Methods are, therefore, required to increase the supply of KELEA to humans and animals. This Application provides a convenient and effective method to help achieve this goal.

### BRIEF SUMMARY OF THE INVENTION

It is discovered that the wearing of containers of KELEA activated water can help restore the prior natural level of KELEA, which was available in humans and animals. It is further discovered that the emission of KELEA by these containers of KELEA activated water can be confirmed using an assay of KELEA mediated weight increase in regular writing paper. It is discovered that the KELEA induced increase in the weight of paper is reversible with electromagnetic radiation.

Basically, the Applicant has shown that in addition to the cellular energy obtained from photosynthesis in plants and from the metabolism of food in plants, animals and humans, living creatures can obtain energy from a natural force that he has termed KELEA (kinetic energy limiting electrostatic attraction). He has further shown that electromagnetic radiation can compete with living creatures for KELEA. The increasing levels of manmade electromagnetic radiation has, therefore, led to a reduction in the availability of KELEA to provide optimal energy support for living creatures. This invention provides a practical means of helping to restore optimal levels of KELEA to humans and animals. It comprises the placing of a containing KELEA activated water adjacent to the skin of an individual human or animal subject. The container of activated water is able to transmit KELEA through the skin to add to the energy of the body's fluids, with resulting functional benefits to the subject. The Applicant has also discovered a way to measure the KELEA emission from the containers of KELEA activated water that are intended for long-term wearing by the subjects.

### BRIEF DESCRIPTION OF THE DRAWINGS

Not Applicable and none included

### DETAILED DESCRIPTION OF THE INVENTION

In a series of publications and patent applications, the Applicant has provided evidence for a source of cellular energy in humans, animals and plants, which is different from that provided by calories in food. The additional cellular energy has been described as the alternative cellular energy (ACE) pathway. A natural force, which the Applicant refers to as KELEA (kinetic energy limiting electrostatic attraction), adds a dynamic or kinetic quality to the fluids of living organisms and can significantly enhance the overall wellbeing of the humans. This added dynamic or kinetic energy can be converted to chemical energy in support of cellular functions and may also directly participate in maintaining the normal electrical activities of cells. Humans mainly obtain KELEA via the fluctuating electrical activities of the brain and probably muscles, including the heart. It can also be obtained from the consumption, inhalation, skin application, or even being in close proximity of KELEA activated water.

The fundamental role of KELEA is proposed to be that of preventing the fusion and presumed annihilation of electrostatically juxtaposed opposite electrical charges. It can do so by imposing a repulsive barrier between the opposing charges. The same energy in fluids can lead to a loosening of the intermolecular electrostatic hydrogen bonding and an increase in volatility and in the internal movements within the fluids. The effect is also seen as a reduction in surface tension of the fluid. The Applicant has devised various assays to reliably measure these changes in water, ethanol and other fluids. Specifically, the volatility of water can be measured as weight loss in closed but not completely sealed containers. Surface tension and internal movements can be monitored by sprinkling fine particles of neutral fed onto the surface of water. In static water, these particles stay on the surface of the water and slowing dissolve into expanding flat, filled colored circles around the particles. In more activated water, the particles pass below the surface and can move in a sideways linear, back and forth manner. The particles tend to aggregate into a small cluster, from which individual particles are repulsed, only to return back towards the cluster.

KELEA binds to separated electrical charges. It can, therefore, bind to dipolar molecules, which by definition have separated positive and negative electrical charges. Certain dipolar compounds can both attract and release KELEA, possibly in an oscillatory manner. Examples of these compounds include humic and fulvic acids, zeolites, mica, several common plant derived materials, and others. When these compounds are placed either into regular water or simply placed close to the water, the water will acquire a measurable level of activation. If water is sufficiently activated, its more separated electrical charges will directly attract KELEA from the environment in a manner similar to water activating dipolar compounds. This allows for the activation of added water, as is practiced in homeopathy or even to water regular water that is placed near to the activated water.

Two recent findings have helped in emphasizing the value of using KELEA activated fluids in supporting the health and wellbeing of humans, animals, and plants. The first was showing that KELEA activated water can activate cellulose containing materials, including paper, cotton fabrics and unprocessed wood. The activation of these compounds was shown by a significant increase in weight of >1 mg/gram, normally within 60 minutes. This observation is consistent with an earlier suggestion that KELEA can add to the weight of compounds. The second observation is that electromagnetic radiation can strip the added KELEA from activated sheets of paper. It is estimated that well over a million-fold increase in the levels of manmade electromagnetic radiation within the atmosphere. Since cosmic rays are comprised of separated electrical charges, mainly protons and electrons, they presumably bring KELEA to the earth. The amounts of KELEA actually reaching the surface of the earth would, therefore, have been reduced by the increasing levels of manmade electromagnetic radiation. The availability of KELEA activated water can help compensate for the lower availability of cosmic ray delivered KELEA to the earth. At issue in this application is the discovery by the Applicant of a convenient, effective, inexpensive, measurable and long-term manner of providing KELEA to humans and animals. This invention deals with this issue.

Over the last several years, the Applicant has had the opportunity to compare different examples of KELEA activated water for their therapeutic benefits. The Applicant trademarked the name Waterceutical™ for KELEA activated drinking water. He has also applied the waterceutical term to KELEA activated water, which is administered intravenously, by inhalation, intranasally, or simply sprayed or otherwise applied to the skin. A novel approach, which is covered in this patent application is placing a flexible container of KELEA activated water or other fluids to the skin of the individual. He is now using the term “wearable waterceutical” to describe containers of KELEA activated water, intended to be placed onto the skin of individuals. After evaluating several types of containers, he chose as a suitable container, a polyvinyl chloride (PVC), mini hot water bottle, of 100 ml capacity that measures 12×11 centimeters. It is produced in Hong Kong by a company called Dragon Sonics and sold as a hot water bottle for infants. These bottles are available from Amazon and other supplies. Similar sized and unlabeled containers are available for bulk purchases from Taizhou Trade and other companies in China. The opening to the bottle is via a screw top. Once the bottle is filled with activated water, the top can be securely closed and rendered airtight by applying either liquid glue or plumber's sealant tape to the thread of the screw before closing. Wedbond brand liquid glue was used with some containers.

Closed, flexible containers with approximately 60 ml of KELEA activated water can be easily inserted with socks or underwear. The screw tops have a hole that can be used to attach the containers to a belt or a neckless. In this way there can be essentially continuous use of the container as an external source of KELEA to the body.

The following studies have validated this approach. First, the containers are an improvement over the prior use of heat-sealed Ziploc bags, that tended to leak over time. Second, the water within closed containers have stayed activated for several years. This was recently confirmed after using the mini hot water bags, which have been worn by the Applicant for several weeks. Indeed, placing the bags within socks or elsewhere on the body provides for repeated jolting of the water, being comparable to the process of succession as used in homeopathy. The economy of being able to continually use the same sample of activated water, rather than drinking, inhaling or spraying KELEA activated water to the skin is obvious, with an expected life time use of single bags being several years. Another supporting aspect of using the containers is the discovery of a relatively simple means of confirming the KELEA activity of a closed container. It was recently found that the weight of a regular sheet of typing paper will increase if placed for a period of time near a container or containers of KELEA activated water. The increase could generally be seen by 30-60 minutes exposure and was in the order of several milligrams per gram of original weight of the sheet of paper (generally about 4.5 grams). An additional discovery was that the increased measured weight of the sheet of paper would be slowly lost by leaving the sheet of paper in the electron balance. This was attributed to the electromagnetic radiation from the electronic balance, since the effect also occurred with other sources of electromagnetic radiation. This system allows for an assessment of the KELEA activity of wearable bags of KELEA activated water. Specifically, a bag to be tested can be placed onto a regular sheet of KELEA non-activated paper and testing the paper for a weight gain of >1 mg/gm within an hour. It can then be shown that the weight gain is reversed by continually weighing the paper in an electronic balance or exposing the paper to other sources of electromagnetic radiation.

Clinically, the Applicant has assessed the relative benefits of repeated spraying of various KELEA activated water i.e., waterceutical products, which he has either produced himself or obtained from different vendors of activated water. These vendors include 1. World Health Advanced Technologies, manufactures of Enercel for intravenous, intranasal, inhalational use or for straying onto the skin. 2. Starchamber products, manufacturer of Vortical Energy Water (VEW) for drinking. 3. Dearborn Technologies, manufacturer of Atoms Water wand to activate drinking water. 4. MI Hope Inc., (a non-profit managed by the Applicant), manufacturer of Glacial Blue, a copper-silver-citrate (CSC) solution, etc. Note that, except for MI Hope Inc. none of the other vendors relate the use of their products to either KELEA or to the alternative cellular energy pathway. All of these products have been found suitable for inclusion into the mini hot water bags, as opposed to the manner of use intended by the vendors of the products. The major advantage of the mini hot water bags has been the continuation of action and a single one-time purchase of usually a set of two “wearable waterceuticals.” Individuals have responded favorably when presented with the alternative of a single versus the need for recurring purchases. Given the argument that essentially everyone should be compensating for the reduced availability of natural KELEA as a result of increased manmade electromagnetic radiation, the long-term use of a set of wearable waterceutical is clearly preferable over transient periods of use. Furthermore, the Applicant has the experience and ability to independently assess the KELEA activating capacity of the wearable waterceutical devices in terms of a transmitted increase in weight of sheets of paper. The Applicant can also easily test for secondary activation of regular water using the devices. This adds another level of reassurance of activity to potential customers.

Concerning the placement of the KELEA activated water containing mini hot water bags, the Applicant has evaluated several locations. The preferred placement has been within the socks at the ankles. This results in near constant agitation of the water while walking, which is comparable to the practice of succession or jolting as practiced in homeopathy. Other placements include being placed between the skin and underwear, strapped to the wrist and placed under the head on a pillow while sleeping.

In addition to hand-sized mini water bottles, other containers can be used for various clinical conditions. These can be miniaturized, as for example, as a device to be placed in the ear canal for treating hearing disorders. Incorporating KELEA activated water containers into the ear pieces of a typical headset can similarly be used. Small, flexible, sealed “blister pack” wearable waterceuticals can be useful for treating small lesions, such as herpes simplex virus infections, aged spots, and others. The sealed containers of KELEA activated fluids can also be much bigger, as in the case of covering large areas of burned skin, or as a skull cap for promoting hair growth. Much larger containers can also be envisioned, such as a KELEA activated water filled mattress. The KELEA activated water filled bags can also be used to activate water for drinking or for spraying onto parts of the body. Any concerns about the leaching of dyes from colored mini water bottles can be addressed by enclosing the bag into a Ziploc outer bag or using a transparent PVC bag.

For many types of animals, a hollow collar can be filled KELEA activated fluid and placed onto the animals' neck. In many disease conditions, the use of KELEA activated fluids in containers positioned onto the skin will be supplemented with additional modes of administration of KELEA activated water.

Transparent PVC containers can be used with the advantage of being able to observe the underlying skin. Transparent PVC containers bags can also be used in conjunction with ultraviolet light illumination of the contained KELEA activated fluid.

Other types of KELEA activated fluids can be used including ethanol containing fluids. appropriately sized KELEA activated fluid bags can be used on animals and on plants. The bags can also be used to activate bulk supplies of water. For many of these other uses, less flexible and more durable PVC or other containers can be used.

In introducing this invention to the general public, the major points being made are 1. As a result of the increasing levels of manmade electromagnetic radiation, there is less available KELEA to supply the alternative cellular energy (ACE) pathway with adequate energy. It is, therefore, of value to obtain an additional, readily, and constantly available source of KELEA. Wearing or attaching a container of KELEA activated water against the skin or exterior surface can be a continuing source of KELEA for humans, animals and plants.

The invention now being described, it will be apparent to one of skill in the art that the findings are reflective of an entirely new paradigm of almost unlimited possibilities. It will also spur new discoveries into the physics of KELEA. Modifications and extensions of the topics included in this application will undoubtedly follow without departing from the spirit and scope of the discoveries.

1. A method of enhancing the alternative cellular energy (ACE) pathway in human and in animal subjects, comprising the long-term placement onto a skin area of the subject, a sealed container of fluid that has been activated by a type of energy that the Applicant refers to as KELEA, being an abbreviation for kinetic energy limiting electrostatic attraction, and which the fluid in the container is able transmit KELEA to the subject and, thereby, able to enhance the subject's ACE pathway.

2. A method of quantitatively assessing the KELEA transmitting activity of a container of KELEA activated fluid by measuring its ability to increase the weight of a sheet of regular paper that is placed onto the container, and further confirming that the increase in weight of the sheet of paper is due to added KELEA by showing that the increase in weight is subsequently reversed during the continuing weighing of the paper in an electronic balance; this being due to the displacement of the added KELEA by the electromagnetic radiation that is associated with the operation of the electronic balance.

3. The method of claim 1 in which the container for use in human subjects comprises a commercially available polyvinyl chloride (PVC) mini hot water bottle that can be easily secured onto different parts of the body.

4. The method of claim 1 in which two containers for use in human subjects are held in place on the skin near the ankles by the wearing of socks or stockings, which confine the KELEA activated water containing item against the skin of each of the ankles.

5. The method of claim 1 in which two containers for use in human subjects are held in place by an item of underwear, which is used to confine the KELEA activated water containing item against the skin.

6. The method of claim 1 in which two containers are composed of clear polyvinyl chloride (PVC).

7. The method of claim 1 in which two containers are composed of water impermeable materials other than polyvinyl chloride (PVC).

8. The method of claim 1 in which the size of the container is miniaturized, such that it can fit into the ear canal in the case of hearing loss; or which can be discretely placed over an area on the skin on which there is an ACE pathway treatable medical condition.

9. The method of claim 1 in which the ACE pathway of an animal can be enhanced by the long-term placement of a hollow collar that contains KELEA activated water to the animal.

10. The method of claim 1 in which the stopper of the mini hot water bottle is coated with a liquid glue or wrapped with thread sealant tape, before being screwed into the bottle containing the KELEA activated fluid in a way to help ensure an air tight sealing of the stopper with the opening in the bottle.

11. The method of claim 1 in which the KELEA activated water is confined to a container other than the mini hot water bottle described in this Application and, which may include a typical headset in which the speakers have been replaced with containers of KELEA activated water.

12. The method of claim 1 in which the KELEA activated water is confined to a container other than the mini hot water bottle described in this Application and, which may include a water filled mattress, pad or sleeping bag.

13. The method of claim 2 in which a positive finding requires a minimal increase in weight of a single 8″×11″ sheet of typing paper of greater than 1 mg/gram and that this increase occurs within 1 hour of placing the sheet of paper onto the container of KELEA activated water.

14. A method of quantitatively assessing the KELEA activity of a container of KELEA activated fluid by measuring the ability of the container when placed into a volume of regular water to lead to the activation of the regular water as determined by measurements of the exposed water for an increase in volatility as reflected in a higher rate of evaporation in closed by not completely sealed containers and/or by the lower surface tension and linear movements of small particles of neutral red dye sprinkled onto the water.
